(19) United States
(12) Patent Application Publication
(10) Pub. No.: US 2015/0066039 A1
(43) Pub. Date: Mar. 5, 2015

(54) CONIC RETRACTION

(71) Applicant: NLT SPINE LTD., Kfar Saba (IL)

(72) Inventors: Tzony Siegal, Moshav Shoeva (IL); Oded Loebl, Tel Mond (IL); Didier Toubia, Raanana (IL)

(21) Appl. No.: 14/390,039

(22) PCT Filed: Jan. 24, 2013
(86) PCT No.: PCT/IB2013/050618

§ 371 Date: Oct. 2, 2014

Related U.S. Application Data
(60) Provisional application No. 61/620,465, filed on Apr. 5, 2012.
(60) Provisional application No. 61/671,816, filed on Jul. 16, 2012.

(51) Int. Cl. A61B 17/02; A61B 17/88

(52) U.S. Cl. CPC A61B 17/025; A61B 17/8897; A61B 17/0218; A61B 2017/0256
USPC 606/90; 606/185

(57) ABSTRACT

A conic retractor for retraction of tissue of a mammal may comprise an at least partially hollow first truncated cone (20); a second truncated cone (30), having a similar apex angle and whose opening is wider than the first truncated cone, may be configured to at least partially enter the first truncated cone and to dilate at least a distal end of the first truncated cone as it is pushed to the distal end of the cone. A third truncated cone (40) can be inserted into the second truncated cone, etc. Cones can be dilated by having a longitudinal slit (29) or overlapping longitudinal wall surface edges, or by the nature of the material the cones are made of.

### FIELD AND BACKGROUND OF THE INVENTION

The present invention generally relates to apparatus and methods for dilation of tissue in a mammal, and more particularly to apparatus and methods for conic retractors.

Distracting tissue, for example intervertebral disc space, involves inserting something into the tissue, whether it is an implant or a distracting instrument or a conduit. In the human body and other mammals, vascular and neural structures lie in the path normally taken by the insertion of these structures.

One common technique currently employed is passing the tip of a K-wire through tissues to the target location—then passing over the wire a succession of tubes of increasing diameter until reaching the optimal bore size. The drawback of this procedure is the ever increasing frictional trauma imposed on the tissue being separated. An additional shortcoming of the ‘tube’ system is that the final cross sectional shape is round.

In certain tissue environments, for instance when the target is the intervertebral disc space, approached from any angle, the above technique is not easily applicable because of the contiguous vital structures like nerves and/or blood vessels that may be damaged in the process.

There is a compelling need for an improved apparatus and method for tissue separation, particularly in spinal surgery, and especially an apparatus and method that minimizes the disturbance of surrounding tissue when inserting dilators, conduits or instruments used for tissue distraction.

### SUMMARY OF THE PRESENT INVENTION

One aspect of the present invention is a conic retractor for dilation of tissue of a mammal, the conic retractor comprising an at least partially hollow first truncated cone; and a second truncated cone, the second truncated cone configured to at least partially enter the first truncated cone and to dilate at least part of the first truncated cone.

A further aspect of the present invention is a method of conic retraction of tissue of a mammal, the method comprising inserting an at least partially hollow first truncated cone into the tissue; and inserting at least part of a second truncated cone into the first truncated cone such that the second truncated cone widens at least part of the first truncated cone.

A still further aspect of the present invention is a method of conic retraction of tissue of a mammal, the method comprising inserting an at least partially hollow first truncated three-dimensional object into the tissue; and inserting at least part of a second truncated three-dimensional object into the first truncated three-dimensional object such that the second truncated three-dimensional object widens at least part of the first truncated three-dimensional object, each of the first and second truncated three-dimensional objects: has a base at a first end; has an opening at a second end as a result of being truncated, at least partially horizontally, at the second end; is wider at the first end than at the second end; gradually widens as one moves along a longitudinal axis of the three-dimensional object from the second end to the first end.

These and other features, aspects and advantages of the present invention will become better understood with reference to the following drawings, descriptions and claims.

### BRIEF DESCRIPTION OF THE DRAWINGS

Various embodiments are herein described, by way of example only, with reference to the accompanying drawings, wherein:

FIG. 1 is an isometric view from the side of a first, second and third truncated cone for a conic retractor showing cone anchors, in accordance with one embodiment of the present invention;

FIG. 2 is an isometric view of the first, second and third truncated cones of FIG. 1 from an angle revealing a longitudinal slit in the first and second truncated cones, in accordance with one embodiment of the present invention;

FIG. 3 is an enlarged isometric view of a truncated cone used in a conic retractor revealing overlapping wall surfaces and including a handle, in accordance with one embodiment of the present invention;

FIG. 3A is an isometric view of a section of a wall of a truncated cone showing the overlapping wall edges, where the overlap encompasses several layers, in accordance with one embodiment of the present invention;

FIG. 3B is an isometric view of a section of a wall of a truncated cone showing teeth and ridges in overlapping wall edges of a wall of a truncated cone, in accordance with one embodiment of the present invention;

FIG. 4 is an enlarged isometric view of a second truncated cone used to dilate another truncated cone, in accordance with one embodiment of the present invention;

FIG. 5 is a side view of a truncated cone of FIG. 4 with an anchoring handle, in accordance with one embodiment of the present invention;

FIG. 6 is an isometric view of a truncated cone used to dilate another truncated cone, in accordance with one embodiment of the present invention;

FIG. 7 is a side view of a truncated cone of FIG. 6 with an anchoring handle, in accordance with one embodiment of the present invention;

FIG. 8 is an isometric view of a dilating truncated cone inserted into another truncated cone, the dilating truncated cone positioned over a starting dilator, in accordance with one embodiment of the present invention;

FIG. 9 is an isometric view of a truncated cone inserted inside a longitudinally overlapping or a split truncated cone that itself is partially inserted in a longitudinally overlapping or split truncated cone, in accordance with one embodiment of the present invention;

FIG. 10 is a rotated view of the truncated cones shown in FIG. 9 revealing a longitudinal overlap or a slit in the truncated cone that is partially inserted into the other longitudinally overlapping or split truncated cone, in accordance with one embodiment of the present invention;

FIG. 11 is a side view of the truncated cones shown in FIG. 9, in accordance with one embodiment of the present invention;

FIG. 12 is a longitudinal cross-sectional view of the truncated cones shown in FIG. 9, in accordance with one embodiment of the present invention;

FIG. 12a is an isometric view of a truncated cone positioned over a cylindrical starting dilator, in accordance with one embodiment of the present invention;

FIG. 13 is a flowchart showing a method in accordance with one embodiment of the present invention;

FIG. 14 shows various schematic horizontal cross-sectional views of truncated cones, in accordance with various embodiments of the present invention;

FIG. 15a is a side view showing a truncated cone with a distal protrusion, in accordance with one embodiment of the present invention;

FIG. 15b is a side view of a truncated cone having a curved annular extension adjacent a distal end of the cone, in accordance with one embodiment of the present invention;

FIG. 16 is an isometric view showing a truncated cone with a second slit, in accordance with one embodiment of the present invention; and

FIG. 17 is a flowchart showing a further method in accordance with one embodiment of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention generally provides an apparatus and method for one or more dilations of cones in tissue of the body, for example in intervertebral disk space. The conic retractor may comprise an at least partially hollow first truncated cone and at least one further truncated cone (called a second truncated cone, or a subsequent truncated cone). Typically, the second or subsequent truncated cone is also at least partially hollow. The second truncated cone may be configured to at least partially enter the first truncated cone and to dilate at least a distal end of the first truncated cone. One or more of the cones to be dilated or widened are dilatable either due to mechanical arrangements (i.e. slits or wraparound edges of the wall surfaces) or are dilatable by the nature of the material that the cones are made of. Typically, a cone that dilates another cone (for example a second truncated cone that dilates a first truncated cone) has the same or similar apex angle to keep pressure on the walls of the cone uniform. The dilation(s) of the first truncated cone, and any further dilation of a second or other subsequent cones, may position the cone to function as a conduit to accept a further dilating element, such as a screw, which then may be used to distract tissue. Alternatively, the conic dilation(s) may themselves effectuate distraction of tissue.

In contrast to prior art methods and apparatuses of dilating tissue, in which a series of tubular containers are inserted in series into the tissue, and as a result trauma may be caused to the tissue from each passage of the container, the conic retractor and method of conic retraction of the present invention may dilate tissue by exerting a force from within a container. This may avoid unnecessary trauma caused by repeated insertions that contact the tissue. In further contrast to prior art methods or apparatuses filed by the Applicant herein, in which dilation may be accomplished from within by a cylindrical or rectangular container, the retractor used in the present invention may comprise a series of cones. The conical shape may facilitate penetration into the tissue. Furthermore, in contrast to other shapes of distracting components used in the prior art for retraction of tissue, the conic retractor of the present invention may afford a gentle and gradual tissue retraction and at least in preferred embodiments may utilize a rounded structure with no irregular or sharp protuberances to impinge on the retracted tissue. In further contrast to the prior art, which may be suitable for retraction of more superficially situated tissue, the present invention may obtain the desired dilation deep within the tissue or organ (an intervertebral disc, for example), for example 90 mm deep, or deep within the surgical site with minimal disturbance of the tissues covering the target. The conic retractor of the present invention, and associated method, may reduce bleeding and may ensure a safe path for repeat introduction of tools into the target. The conic retractor and associated method of the present invention may protect surrounding critical structure (nerves, for example). In still further contrast to prior art methods and apparatus of tissue distraction using an implant, in which an overall shape of the distracting instrument changes, for example from straight to curved, the conic retractor of the present invention may achieve distraction while maintaining the overall shape of the dilated and dilating cone. This may reduce the amount of disturbance of the surrounding tissue. In yet still contrast to prior art where the entire length of the distracting element has to flex or change shape to either position this element and/or to perform the distraction, which may result in disturbance to tissue throughout the length of the element used for distraction of the tissue, with the conic retractor of the present invention, retraction may be accomplished even though only the part of the conic retractors that will be active in the retraction need be dilated. For example, only the distal part of the first truncated cone needs to be widened in order to effectuate the retraction. This may be because in some preferred embodiments the slit or the area of overlapping wall edges may be limited to the distal tip of the cone. In still further contrast to the prior art, in which a retracting element has to first be flexed or changed in shape merely to position the distracting device into position to perform the distraction, which change disturbs surrounding tissue, at least in some preferred embodiment, the conic retractor of the present invention may be positioned in place for distraction without any change in shape and the widening/dilation of the distal tip of the cone may itself simultaneously effectuate the distraction. In still further contrast to prior art methods, the method of conic retraction of the present invention may make it easier to position the distracting element into place for the retraction due to the gradually widening outer surface of the cone from its “apex end” to its base. In contrast to the prior art, in which only a limited number of instances of retraction can realistically be performed without traumatizing the surrounding tissue, the apparatus and method of the present invention may allow repeated subsequent retractions without further disturbance, since a subsequent truncated cone can be pushed inside a previously inserted cone repeatedly. In contrast to the prior art, if the tip of the cone used in the conic retractor of the present invention is pressed against a hard surface and then dilated, it may typically sweep away most tissue with little danger to critical vascular or neural structures.

The principles and operation of an apparatus and method for a conic retractor according to the present invention may be better understood with reference to the drawings and the accompanying description.

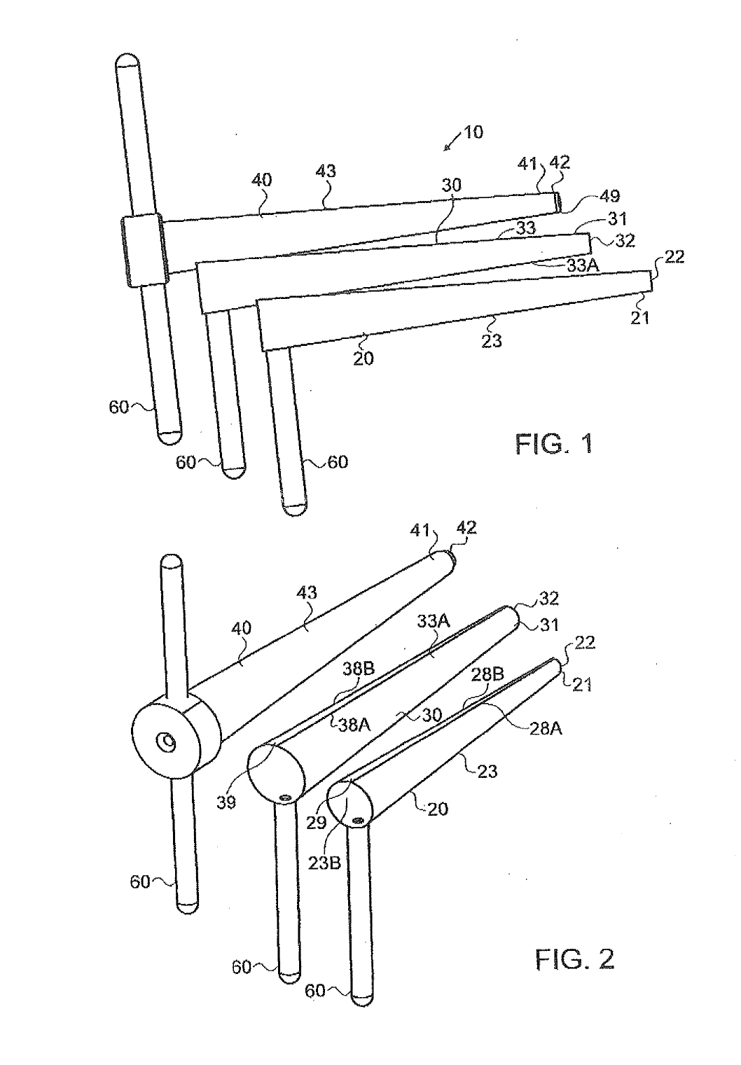

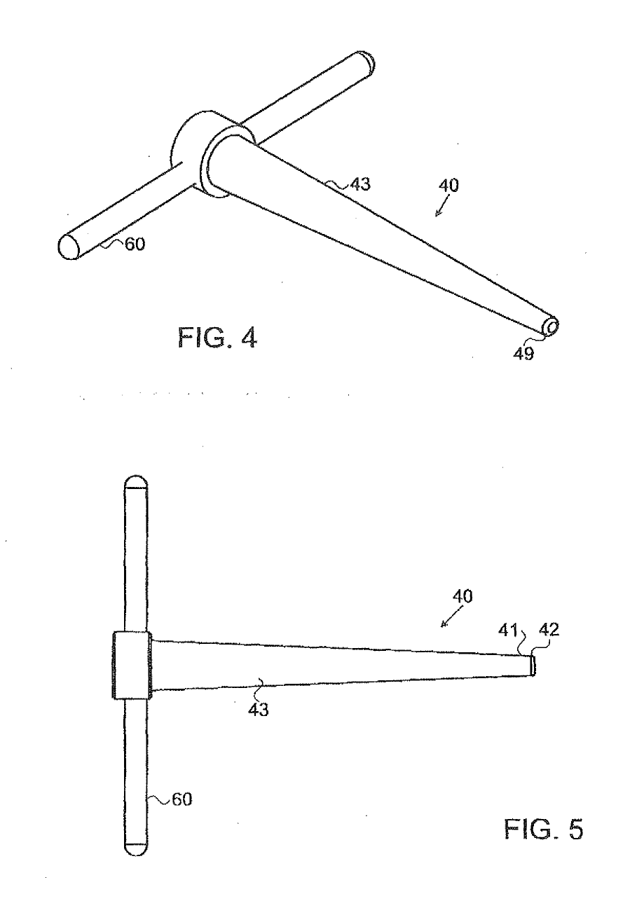

A “truncated cone” is a cone lacking an apex (whether the apex has been physically cut off or never existed) and terminating in a plane or in a partial plane or straight or curved surface, whether or not the plane or partial plane or surface is parallel to the base. The truncation may be at a partial plane rather than at a plane since the cone may be hollow or at least hollow at the relevant longitudinal portion of the cone, i.e. at the distal end. Furthermore, as shown by the distal end of the third truncated cone 40 shown in FIG. 1 and the cone shown in FIG. 4, the truncated cone 40 is still within the definition of a “truncated cone” even though it has been modified to include a rounded area 49 at the tip between the plane or partial plane and the walls 43 of the cone. FIG. 15b depicts a truncated cone 20 having a curved annular extension 77 adjacent the distal end 22. The opening of the cone at distal end 22 is demarcated by points 77a, 77b. This cone shown in FIG. 15B is an example of a truncated cone used in the conic retractor/method of the present invention not necessarily terminating in a flat plane or partial plane. A truncated cone shall also not cease to be within the definition of a “truncated cone” if its tip at or near the plane or partial plane of truncation is modified by addition of bumps or ridges or small arced edges, or by the subtraction of recesses or grooves, adjacent or on the plane or partial plane.

The plane or partial plane of truncation intersects the cone and is referred to herein as the “truncated surface”. If the truncated cone is hollow at its distal end, then due to the thickness of the walls of the cone, the truncated surface may include an inner loop (typically although not necessarily closed since there could be a gap in the wall of the cone) at the inside wall surface of the cone and may an outer loop at the outer wall surface of the cone. The outer loop or the inner loop may be discontinuous if there is a gap in the walls of the cone. The opening at the distal end of a hollow truncated cone may be called its inner width since it excludes the thickness of the walls.

The term “truncated cone” as used herein includes truncated cones which have been modified by adding material as a “distal protrusion” (beyond the plane or partial plane of truncation). For example, FIG. 15a depicts a side view of a first truncated cone 20 comprising a truncated cone with a truncated surface not parallel to the base 24 such that the distal end 22 of the cone is at the distal plane 22p that intersects the cone and comprising a distal protrusion 25 more distal than the distal end 22. This modified truncated cone is included in the definition of truncated cones.

The truncated cone of the present invention therefore need not be round, may not come to a point, and may merely form a conic portion of a larger object whose overall shape may not be a cone or appear to be a cone.

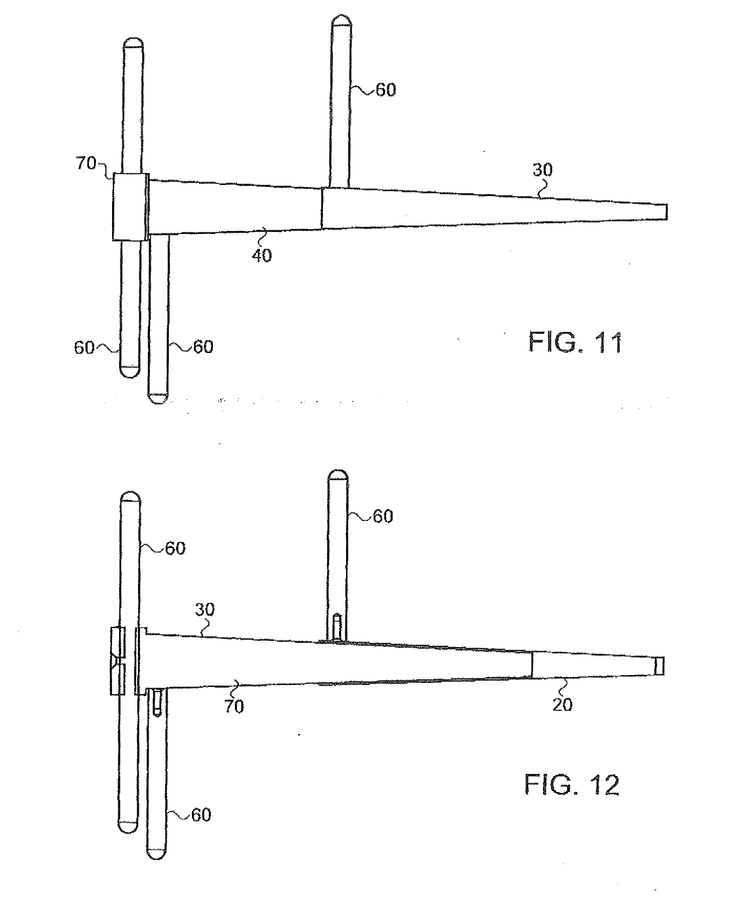

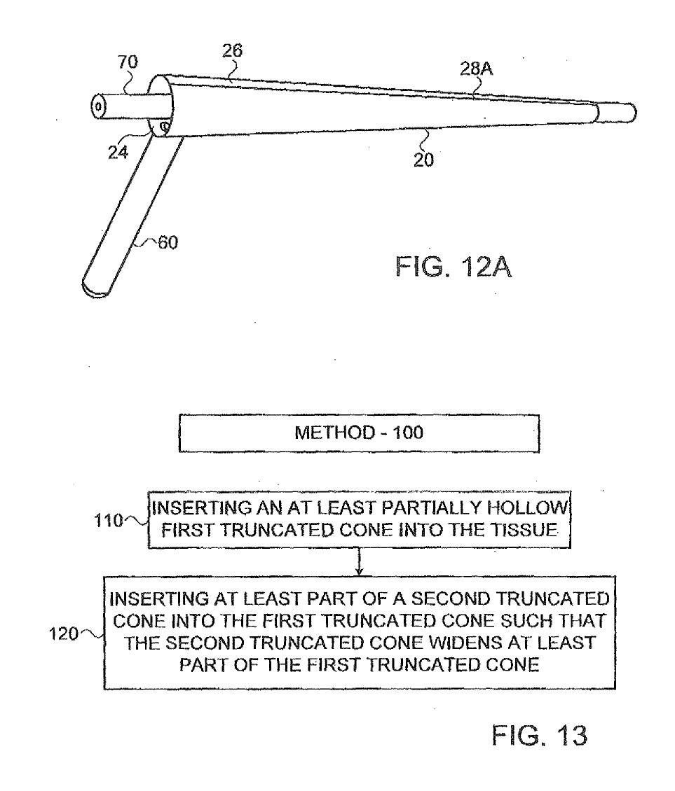

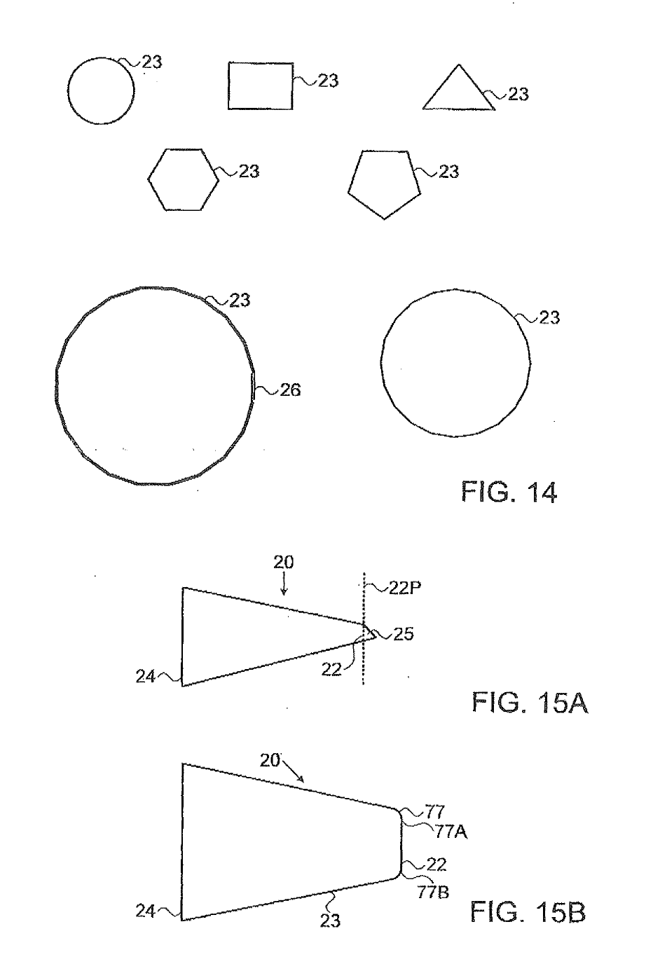

In one particularly preferred embodiment, the truncated cones of the conic retractor of the present invention are circular in cross-section so that the forces from the walls of the second cone against the walls of the first cone, at least in certain sections of the cone, for example the distal end, are substantially equal due to the symmetry of the cross-section (i.e. a circle). In other preferred embodiments, see FIG. 14, the cross-section of the cone is polygonal, which is another symmetrical cross-section, at least at its distal end. In other preferred embodiments, the cross-section of the cone, at least at a distal end, is elliptical or oval or is otherwise arcuate or looped. In all cases, the cross-sections being referred to are horizontal cross-sections (not to be confused with a longitudinal cross-section like that shown in FIG. 12). Furthermore, in a preferred embodiment, the horizontal cross-section of each of the truncated cones (for example first truncated 20, second truncated 30 and third truncated cone 40) used in the conic retractor or method of the present invention is the same, or virtually the same.

For truncated cones of elliptical cross-section (or another cross-section where one non-longitudinal dimension or width is larger than the other non-longitudinal dimension), at least at the distal end, the second truncated cone should preferably be wider than the first truncated cone in both non-longitudinal dimensions (i.e. in both dimensions of a plane parallel to the base). In other preferred embodiments, the second truncated cone is wider than the first truncated cone in at least one of the two non-longitudinal dimensions.

The term “maximum width” is a term used in relation to truncated cones having symmetrical cross-sections (or portions of a truncated cone that has a symmetrical cross-section). For example, in the case of a truncated cone (or a relevant portion of a truncated cone) having a circular cross-section, the maximum width simply means the diameter. In the case of a truncated cone of polygonal cross-section (or a relevant portion of a truncated cone having a polygonal cross-section), the maximum width may or may not bisect the polygon into equal areas.

In this patent application the term “distal” refers to being distal in relation to the base of the cone spoken of. The “distal end” of a truncated cone (whether the cone is of symmetrical cross-section or not) is defined to be at the truncated surface if the truncated surface is parallel to the base. If the truncated surface is not parallel to the base of the truncated cone then the “distal end” of the cone is defined to be at the part of a “distal plane” that intersects the cone, where the term “distal plane” refers to the plane parallel to the base that intersects the part of the truncated surface most proximal to the base.

The term “distal protrusion” shall refer to portions of a truncated cone, if any, that are more distal than the “distal end”. The term “distal tip” shall refer to a portion or area of the truncated cone within X units of axial distance (toward the base) from the “distal end” of the truncated cone. If the units are centimeters, for example, one can specify that the distal tip includes the last or most distal one centimeter, two centimeters, etc. of the truncated cone. In all cases, the term “distal tip” shall not be so long that the X units of distance reach half the axial height of the truncated cone (excluding any distal protrusion).

The “maximum width” at a distal end of a truncated cone in an area of the cone that is of symmetrical cross-section (circular or polygonal) is as follows: if the truncated surface of the truncated cone is parallel to the base of the truncated cone, the maximum width is the “maximum width” of the truncated surface. If the truncated surface is not parallel to the base of the truncated cone then the maximum width is the maximum width of the cone at the “distal plane” (as defined herein).

For truncated cones that are hollow along at least part of the length of the cone, the thickness of the sheet or wall of the at least partially hollow cone will vary according to the conditions presented by the work or by the tissues of the organ (of the human or other mammal) in which the apparatus or method of a conic retractor of the present invention takes place. The material from which the first truncated cone (or subsequent cones) is made may influence the thickness of the cone. For example, in many instances, metals allow thinner sheets or walls (sometimes called “mantles”). The material that the cone is produced from may vary widely according to the intended use and expected pressure on the cone during the dilation process. Materials employed in the cones of the present invention may range from plastics to metals, to shape memory alloys and materials, to composite materials—and any other materials or compounds which exhibit the combination of sturdiness, flexibility and biocompatibility required for the function of dilation. The material employed in the cones preferably has a low coefficient of friction so that relative motion between the cones (or insertion of another device into the cones) does not require a large force.

Note that if the walls of the cone have non-negligible thickness, the “maximum outer width” at the distal end of the truncated cone is defined to be the maximum width at the outer wall surface of the truncated cone and the “maximum inner width” at the distal end of the truncated cone is defined to be the maximum width at the inner wall surface of the truncated cone.

The maximum width at the distal end can also be extrapolated to refer to the “average maximum width” within X units of distance (toward the base) from the distal end, and this average maximum width would just be the average of the maximum widths across the X units of distance. In addition, the X units of distance from the distal end toward the base are measured along the cone's axial height. If the cone has walls of non-negligible thickness, the “average maximum outer width” at the distal end of the truncated cone is defined to be the average of the maximum widths at the inner wall surface of the cone through the X units of distance (axial height) from the distal end and continuing toward the base. Similarly, the “average maximum inner width at the distal end of the truncated cone is defined to be the average of the maximum widths at the inner wall surface of the cone through the X units of distance (axial height) from the distal end and continuing toward the base.

Preceding the terms “maximum width”, “average maximum width”, “maximum inner width”, “maximum outer width”, “average maximum inner width”, “average maximum outer width” with the word “first” or with the word “second” (or the word “third”, etc.) simply indicates that the respective term applies to the first cone or to the second cone (or third cone, etc.), as the case may be. For example, the first maximum width refers to the maximum width of the first cone. The second maximum width refers to the maximum width of the second cone. The first maximum inner width refers to the maximum inner width of the first cone; the second maximum inner width refers to the maximum inner width of the second cone; the first maximum outer width refers to the maximum outer width of the first cone; the second maximum outer width refers of the maximum outer width of the second cone. The first average maximum width refers to the average maximum width of the first cone. The second average maximum width refers to the average maximum width of the second cone. The first average maximum inner width refers to the average maximum inner width of the first cone; the first average maximum outer width refers to the average maximum outer width of the first cone. The second average maximum inner width refers to the average maximum inner width of the second cone; the second average maximum outer width refers to the average maximum outer width of the second cone.

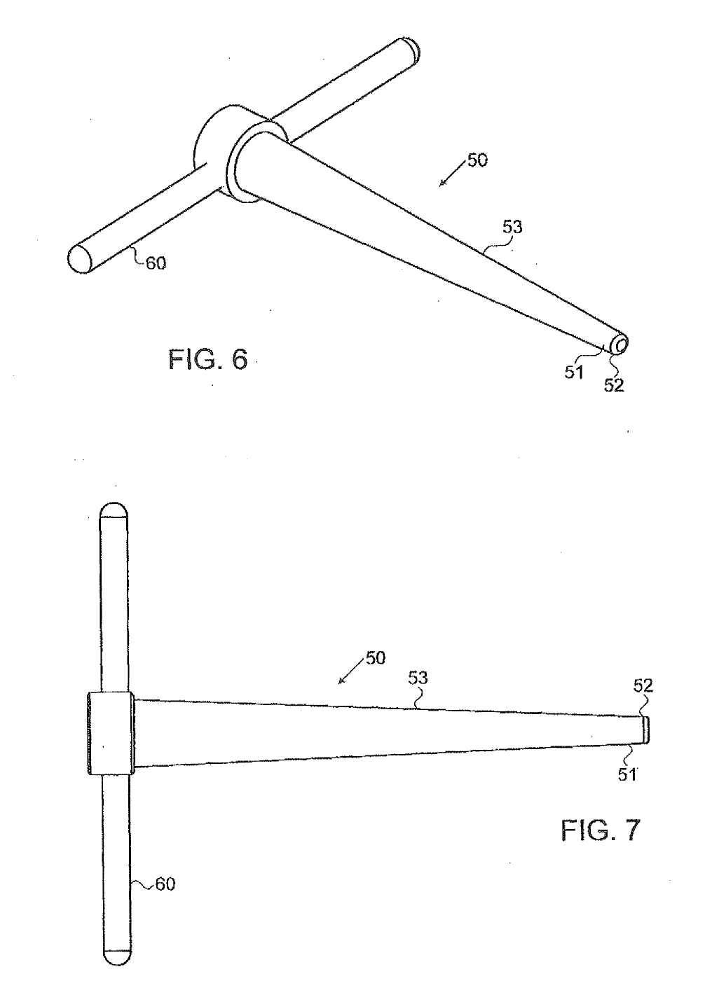

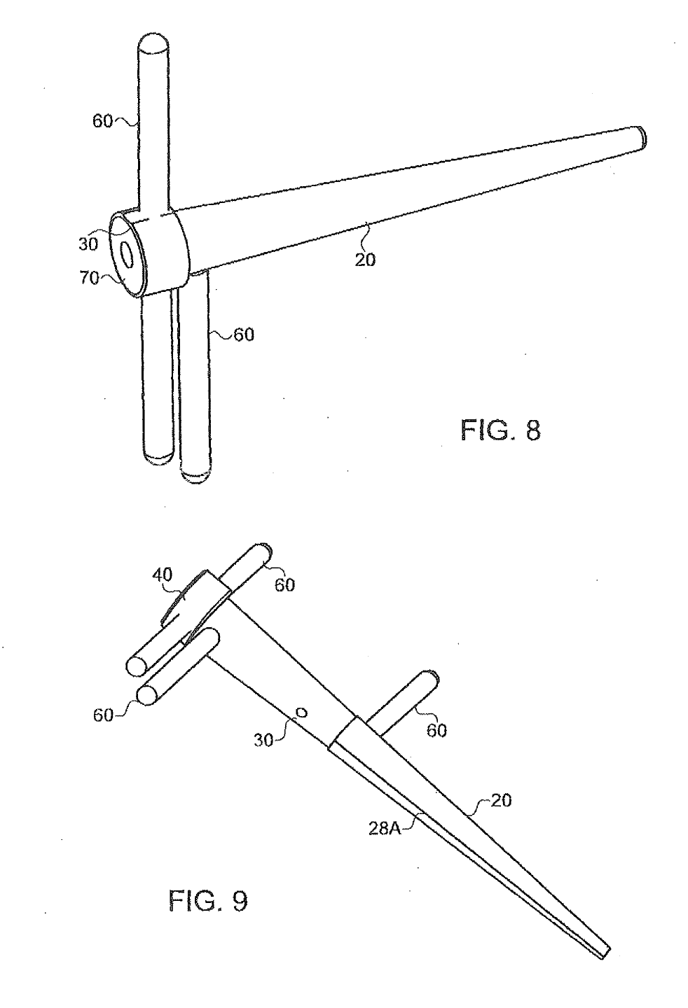

As shown in FIGS. 1-12 generally and in particular in FIG. 1, a conic retractor 10 is shown that may be for retraction of tissue of a mammal, such as a person. Conic retractor 10 may comprise an at least partially hollow first truncated cone 20 having a base 24 and a second truncated cone 30 having a base 34. First truncated cone 20 may be hollow or may be sufficiently hollow to allow insertion of at least a distal end 32 of second truncated cone 30. Moreover, first truncated cone 20 may be sufficiently hollow to allow first truncated cone to slide over a starting dilator into the tissue of the mammal.

The term “retractor” or “retraction” is the preferred term used herein to denote separation of the tissue. Nonetheless, the preferred term used herein for expansion of the cones (from within) used in the conic retractor or method of the present invention is “dilation”. However, it should be understood that the word “dilate” or “dilation” is intended to refer to expansion and there is no suggestion that the dilation is in some way limited to expansion of only a rounded wall/surface of the truncated cone. The dilation applies equally to expansion of walls or surfaces having non-rounded cross-sections, for example those of square or polygonal cross-sections. Furthermore, in many preferred embodiments, the dilation of the cone may directly effectuate the retraction of the tissue. Hence, tissue retraction may on occasion be referred to as tissue dilation, although this is not the preferred terminology.

The dilation principle of the present invention may involve introduction of the next cone into the cone already deployed at the requested site. For the dilation to take place, the already introduced cone must present a tip that can widen its circumference. The sequential increase in the girth of the tip of the cones may be obtained by configuring the cone such that the sharp end of each sequential cone is shortened, by an amount, say 25 mm, and by then adding 25 mm in length at the base-end of this cone. Thus in a preferred embodiment the length of all cones used in the conic retractor may be identical but they may differ by the size of their opening at their tips and by the size of their bases, in each case being sequentially wider.

In other words, in one preferred embodiment, the structure of the second truncated cone 30 may be obtained by starting with a truncated cone that is longer than the first truncated cone 20, for example by twenty-five millimeters (this number is purely an example), but has the same shape and apex angle as the first truncated cone. That longer truncated cone may then be further truncated by twenty-five mms at its sharper “apex end” parallel to its base such that the resulting truncated cone (i.e. the second truncated cone 30) has an axial height equal to the axial height of the first truncated cone 20. Typically, a second truncated cone 30 formed this way may be at least partially hollow and may be as hollow as the first truncated cone 20. In addition, such a second truncated cone may perform a function within the tissue similar to that of the first truncated cone, in that a third truncated cone 40 can then be inserted into it to widen the second truncated cone 30.

The term “subsequent cones” refers to truncated cones in the conic retractor or method of the present invention labeled with a higher ordinal number than a previously referred to cone (for example after referring to the “first truncated cone” “subsequent” cones refer to the second, third, fourth, etc.). Conversely, “previous” or “previously inserted” cones refer to truncated cones in the conic retractor or method of the present invention labeled with a lower ordinal number than a subsequent cone (for example the “first truncated cone” is “previous” to a second truncated cone and a “second” truncated cone is previous to a “third” truncated cone, etc.).

Accordingly, the second truncated cone 30 may be wider than the first truncated cone 20. Second truncated cone 30 may be wider than first truncated cone 20 at their respective distal tips 21, 31, or in some preferred embodiments, at all comparable points (at a given distance of axial height from their respective distal ends) on their distal tips 21, 31 (i.e. up to half the axial heights). In a preferred embodiment, second truncated cone 30 is wider than first truncated cone 20 at all comparable distances (using axial height) from the respective base, or from the respective distal ends 22, 32.

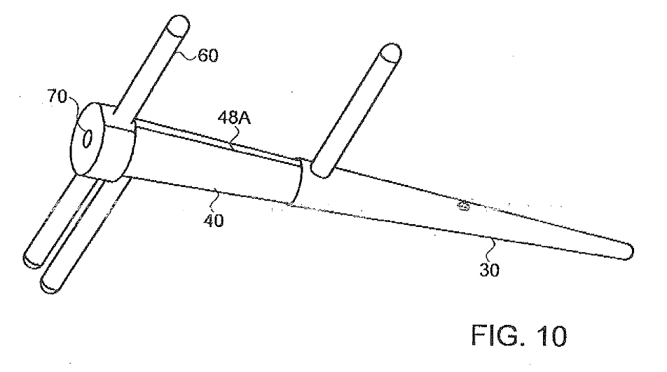

Use of the conic retractor 10 may in some preferred embodiments involve first inserting a sharp thin rod, called a guide wire, at the preferred depth of the tissue, controlled manually or by robot. The depth of the rod may be determined by having the rod measured or by means of a c-arm (i.e. an x-ray/fluoroscopy machine). Preferably, a cylindrical (pencil-like) dilator having a central canal or bore, the dilator preferably having a blunt tip, and having a diameter of for example up 8 mm, may be passed over the guide wire to reach the annulus or outer border of the tissue structure, which tissue structure may for example be a disk of a spine of a human). This starting dilator 70 (FIG. 10) may have an electrode incorporated therein, which is used for nerve detection in the vicinity of the disc or other site that was penetrated into. The starting dilator is a “dilator” in the sense that its insertion necessarily involves a further dilation of the space made in the tissue by the guide wire. Alternatively, instead of the starting dilator being inserted after the guide wire, at the time of insertion of the guide wire (thin rod), the guide wire may be enclosed in a close fitting cylinder which accompanies the rod to the intended destination. The sharpened end of the thin rod may protrude slightly through the end of the starting dilator/cone to ensure smooth tissue or work penetration. In certain preferred embodiments, there can be a second or further starting dilator(s), if a very gradual increase in diameter from the guide wire diameter to the inner diameter of the first cone is desired.

In certain preferred embodiments, for example see FIGS. 10-12, the starting dilator 70 may be conic instead of cylindrical as long as it narrowest diameter allows it to be passed over the guide wire. Even if the starting dilator 70 is conic, though, it still may be distinguished from the conic dilators (20, 30, 40, etc.) of conic retractor 10 in that starting dilator (the first dilator to be placed over the guide wire) is truncated and may allow further truncated cones to be inserted within in (and in that the starting dilator would normally be removed whereas conic dilators 20, 30, 40, etc. of conic retractor 10 may or may not be removed). However, if the starting dilator 70 is conic and truncated, and also meets the other requirements of a first truncated cone in that it allows further cones to be pushed inside it for example by at least its distal end having a longitudinal slit (or by at least its distal end being made of appropriate material), then it may be considered the first truncated cone that forms part of conic retractor 10 and that has been labeled “20” in this patent application.

Once the starting dilator has reached its destination in the tissue of the mammal, its position may be controlled in most cases by an x-ray. The sharp thin rod (guide wire) must contain a radio-opaque quality to be discernible on the x-ray and in most cases is made of metal.

The conic retractor may now be inserted. For example, a first truncated cone 20, oriented such that the sharper end of the cone 20 enters first and the base enters last, may be slid down over the external surface of the starting dilator to reach the site, which site may be the lateral aspect of the annulus of a disc (assuming penetration is lateral). The first truncated cone 20 may be slid down such that the inner wall surface 23b of first truncated cone 20 slides over the external surface of the starting dilator (see FIG. 12A which shows a conic dilator 20 that has been slid over a cylindrical starting dilator 70). Then, a second truncated cone 30 may be pushed through the inside of the first truncated cone but such that the inner wall surface 33b of the second truncated cone slides over the external surface of the starting dilator. Since the first truncated cone 20 may have a longitudinal slit or may have overlapping edges of the wall surface, this insertion of the second truncated cone 30 may dilate the first truncated cone 20 which may further dilate the tissue, for example by an incremental amount. Then, a third longitudinally split truncated cone 40 may be inserted through the inside of the second truncated cone 30 but such that the inner wall surface 43b of third wall 43 (FIG. 1) of the third truncated cone 40 slides over the external surface of the starting dilator. Again, since the second truncated cone 30 may have a longitudinal slit or may have overlapping edges of its wall surface, this insertion of the third truncated cone 40 may dilate the second truncated cone 30 which may dilate the tissue further, for example by an incremental amount. The “last” cone (in terms of order of insertion) of the conic retractor may optionally lack a longitudinal slit or overlapping wall edges (since being “last”, it itself will not be further dilated, although in certain embodiments it may nonetheless have such a slit or overlap in order to expedite insertion or removal).

Each of the truncated cones may have an electrode incorporated therein, which is used for nerve detection in the vicinity of the distal tip of the device or in the vicinity of the disc or other site that was penetrated into. In addition, each of the cones may have an edge lighting mode, which is a source of light that is positioned in proximity to the proximal edge of a cone such that the rays of light enter the thickness of the cone material and pass or travel distally through the cone and out of the distal edge, to illuminate the relevant tissue to offer good and visible exposure for the surgeon. The guide wire, which may for example be a K-wire, may be positioned in the site of the disc or other site penetrated into and may be removed when diskectomy tools are passed into the disc. The last cone to dilate the soft tissue, which may be third truncated cone 40 (or in other preferred embodiments could be a second truncated 30 or a fourth (or greater) truncated cone 50) may function as a conduit assuming the guide wire and starting dilator has been removed and (assuming previous conic dilator(s) have also been removed) as a protector of the surrounding soft tissue against possible damage by the passing of instruments through the last cone into the site. In fact, any cone that has been dilated (i.e. the first truncated cone 20 and/or the second truncated cone 30 and/or subsequent cones if applicable) may be removed as unnecessary since the force of dilation is now being supplied by the dilating cone inside it, which is wider, at least at the relevant distal tip.

First truncated cone 20 may comprise a conical wall, which shall be referred to as first wall 23 since it is the wall of the “first” truncated cone 20. First wall 23 may have an outer wall surface 23a and (since first truncated cone 20 is at least partially hollow) may have an inner wall surface 23b. Second truncated cone 30 may have an outer wall surface 33a. If second truncated cone 30 is hollow, which it preferably is, then second wall 33 of second truncated cone 30 may also have an inner wall surface 33b.

Second truncated cone 30 may be configured to at least partially enter the first truncated cone 20 and to widen or dilate at least part of first truncated cone 20, or in some preferred embodiments, to widen or dilate at least a distal end of the first truncated cone 20, or in some preferred embodiments to widen or dilate the entire (axial) length of first truncated cone 20, or in other preferred embodiments to widen or dilate a longitudinal portion of the first truncated cone 20 that has a longitudinal slit, or in other preferred embodiments, to widen or dilate a longitudinal portion that has overlapping wall edges. Second truncated cone 30 may be configured to at least partially enter the first truncated cone and to dilate at least the distal end of the first truncated cone when an outer surface 33a of second wall 33, at a distal end of the second truncated cone, presses against an inner surface 23b (FIG. 2) of first wall 23 at the distal end of the first truncated cone. The same may be true of third truncated cone 40—it may be configured to at least partially enter second truncated cone 30 and widen or dilate at least part of second truncated cone 30, or in some other preferred embodiments, to widen or dilate at least a distal end, an entire length, or a longitudinal portion having a longitudinal slit or overlapping wall edges. The same may be true of subsequent truncated cones in relation to the truncated cone configured to be adjacent it and previous in ordinal number.

The truncated cones may be configured such that the dilation or widening of first truncated cone 20 (or of second or third or further truncate cones) may occur in set increments, for example dictated by the increase in the diameter of the openings (i.e. the distal tip) of the first truncated cone (or other cone).

In certain preferred embodiments, as a result of the truncation, first truncated cone 20 may have an open space at its distal end, whose size, for example depending on the embodiment, may be 5 mm, 6 mm, 7 mm, 8 mm, 9 mm, 10 mm, 11 mm, 12, mm, 14 mm, 17 mm, 20 mm, 25 mm, 30 mm or 40 mm, or other amounts. If the truncated cone has a circular cross-section at the distal end, the size of the opening is the inner width of the truncated cone at the distal end. If the truncated cone is not circular in cross-section at the distal end, the size of this opening is described by its maximum width. In considering the size of the opening at the sharp end (i.e. the narrower end which is at the distal end) of the cone, any distal protrusions beyond the distal end of the truncated cone are ignored.

In some preferred embodiments, a first wall 23 of first truncated cone 20 may have a non-negligible thickness, purely by way of example one millimeter. A second wall 33 of second truncated cone 30 (or a third wall 43 of third truncated cone 40, etc.) may have a non-negligible thickness, purely by way of example also one millimeter. In order for conic retractor 10 (FIG. 1) to effectuate the retraction, first truncated cone 20 may have a first maximum inner width at a distal end of first truncated cone, and second truncated cone 30 may have a second maximum outer width at a distal end of second truncated cone 30 such that the second maximum outer width is larger than the first maximum inner width.

In preferred embodiments, first truncated cone 20 (or subsequent truncated cones 30, 40, etc.) may have a first average maximum inner width in a distal tip defined to be within a particular distance, for example one centimeter (the distance being measured against the axial height of the cone), of a distal end of the first truncated cone, wherein the second truncated cone has a second average maximum outer width in a distal tip defined to be within a particular distance, for example one centimeter (measured against the axial height of the cone), of a distal end of the second truncated cone, and the second maximum outer width is larger than the first average maximum inner width. In still other preferred embodiments, the first truncated cone has a first average maximum inner width in a distal tip defined to be within two centimeters of a distal end of the first truncated cone, wherein the second truncated cone has a second average maximum outer width in a distal tip defined to be within two centimeters of a distal end of the second truncated cone, and the second maximum outer width is larger than the first average maximum inner width. In yet still other preferred embodiments, the first truncated cone has a first average maximum inner width in a distal tip defined to be within three centimeters of a distal end of the first truncated cone, wherein the second truncated cone has a second average maximum outer width in a distal tip defined to be within three centimeters of a distal end of the second truncated cone, and the second maximum outer width is larger than the first average maximum inner width. One centimeter, two centimeters and three centimeters are merely illustrative of a distance from the distal end. Other preferred embodiments may use 1, 2, 3, 4, 5, 6, 7, 8, 9, 10, 15, 25, 35, 40, 50, 55, 60, 70, 80, 90, or 100 millimeters or still other distances as the distance from the distal end over which the average width or average maximum width is measured, and this may be applicable to the first truncated, the second truncated cone or both. Typically, the average width is measured over equal distances in the first and second (or third or further) truncated cones.

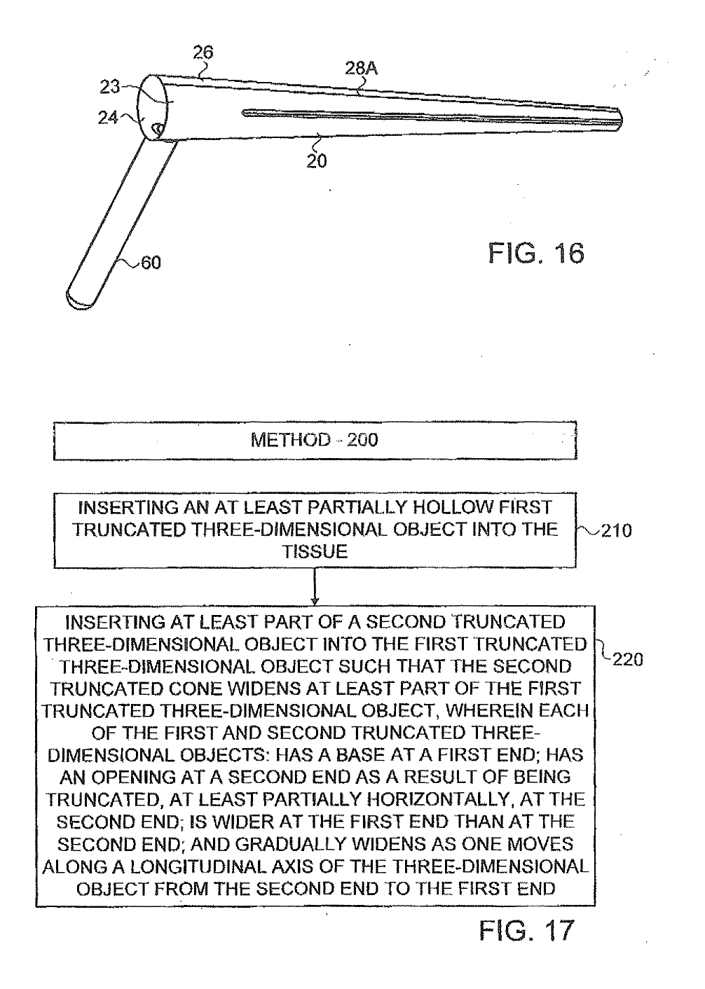

A first wall 23 of the first truncated cone 20 may have a first longitudinal cut, which is referred to as a slit 29, in at least a distal end 22 of the first wall 23, and preferably throughout the length or axial height of the cone. The term “slit” does not preclude an opening wider than a narrow slit, although in one preferred embodiment, slit 29 may be narrow. For example, in certain preferred embodiments, the slit 29 (although this is not the typical definition of term “slit”) may comprise a gap that is not only not thin but is up to one-third of the circumference of the cone (as measured prior to any dilation of the cone). In some preferred embodiments, slit 29 may extend from the distal end 22 (or if there is a distal protrusion it may extend beyond the distal end from the most distal edge of that distal protrusion) all the way to the base, or in other preferred embodiments all the way to a point adjacent or near the base. A cone that has a longitudinal slit is sometimes referred to herein as being longitudinally split. The slit 29 along the first truncated cone 20 (or along subsequent cones such as second truncated cone 30), from the apex to the base can take many shapes: a straight regular cut of the same width along its length, a cut with non-parallel lines, a jagged cut composed of parallel cut-lines, a jagged cut composed of non-parallel cut-lines, curved, arched zigzagged or other. Furthermore, as shown in FIG. 16, first truncated cone 20 (or a subsequent cone) may have a second slit 27, for example a slit that does not extend to either the proximal or distal end of the cone.

In one preferred embodiment, as shown in FIG. 2, slit 29 may separate a first edge 28a of the first wall 23 from a second edge 28b of the first wall 23. The slit 29 may extend longitudinally through one centimeter or in other preferred embodiments through one tenth or one quarter or up to one half of the axial length of the first truncated cone 20. In one preferred embodiment, the slit 29 extends throughout the entire length of the wall of the cone.

The other truncated cones used in the method or apparatus (conic retractor) of the present invention may also include a slit. For example, in some preferred embodiments of conic retractor 10, a second wall 33 (which is called “second” because it is a wall of the “second” truncated cone) of the second truncated cone 30 may have a second longitudinal slit 39 in at least a distal end 32 of the second wall 33 or through one centimeter or other distances mentioned regarding slit 29. Actually, in most preferred embodiments, all truncated cones of the conic retractor or method of the present invention may have longitudinal slits, except that the last truncated cone may optionally not need a longitudinal slit.

It is emphasized that the various descriptions in this patent application of the embodiments for the structure of first truncated cone 20, or for the structure of the second truncated cone 30 used for dilating first truncated cone 20, apply equally to the embodiments for the structure of third truncated cone 40 since third truncated cone may be used in turn to dilate second truncated cone 20. The same applies to any fourth or further truncated cone. Since the very last cone need not be dilated, such last cone (whether that last cone is the second, third or fourth or subsequent truncated cone) may optionally not have any of the following: (i) a longitudinal slit, (ii) an overlap, (iii) being made of a dilatable material.

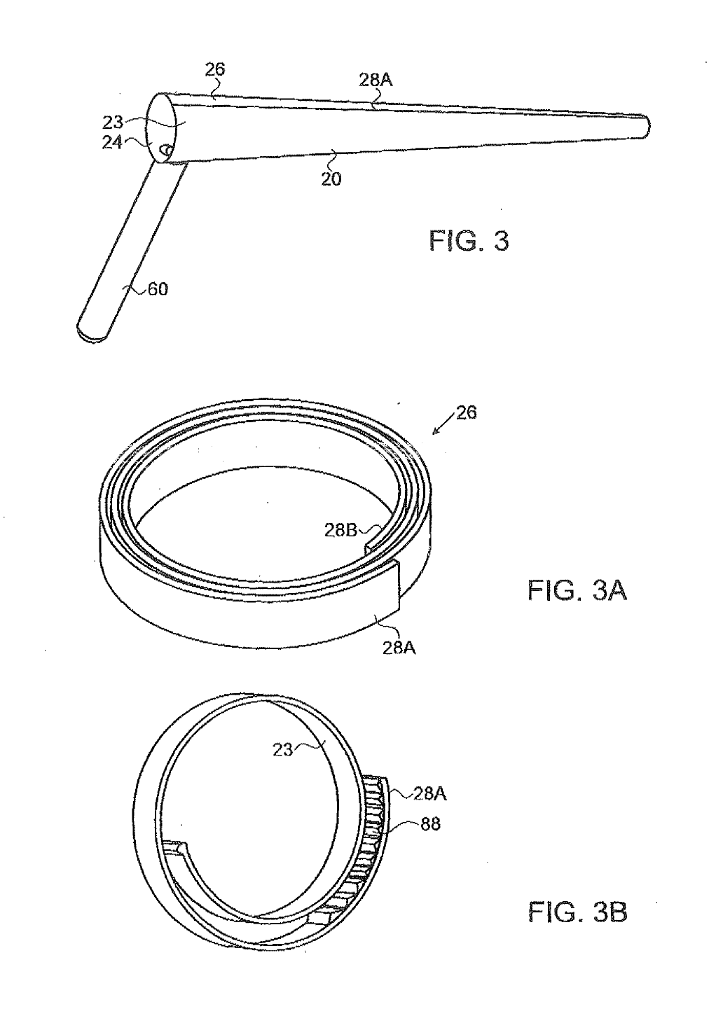

As shown in FIG. 3, instead of a longitudinal slit, in some preferred embodiments, the first edge 28a and second edge (not shown) of the first wall 23 of the first truncated cone 20 may overlap, even generously. It is noted that even the embodiment having the overlap also presupposes a longitudinal separation except that the edges are not separated by a slit or gap but rather overlap. As seen from FIG. 3A, in some preferred embodiments, the overlap 26 of first wall 23 may extend through multiple circumferences of the cone, for example two or three overlapping layers (i.e. circumferences of the wall 23). In some preferred embodiments, the overlapping wall edges 28a, 28b may appear at least at a distal end 22 of the first wall 23. The overlapping edges 28a, 28b may extend longitudinally through one centimeter of an axial length of the first truncated cone 20 or in other preferred embodiments through one tenth or one quarter or up to one half of the axial length of the first truncated cone 20. In some preferred embodiments, a second wall 33 of the second truncated cone 30 may have overlapping wall edges 38a, 38b in at least at a distal end 32 of the second wall 33 or through one centimeter of the axial length of the second truncated cone 30 or other distances or percentages of length mentioned regarding slit 29. The overlap may extend through multiple circumferences of the second truncated cone 30, as shown in FIG. 3A. In some preferred embodiments, the overlapping wall edges are not continuous and may have gaps. FIG. 10 shows an overlapping wall edge 48a of a third truncated cone 40.

Overlap 26 may, in certain preferred embodiments, be a variable overlap. As seen from the bottom two figures of FIG. 14, a first wall 23 of first truncated cone 20 (or walls of subsequent truncated cones 30, 40, etc.) may have a polygonically shaped cross-section (throughout its length or for example at a distal end) comprising for example twenty sides. In certain preferred embodiments, the overlap 26 may be variable and may comprise one or more side-lengths, for example one side-length as shown in FIG. 14. In certain versions, incremental expansion of overlap 26, by for example a side-length or a side length multiple of two or three or more, may increase the number of sides of the resulting polygon in the cross-section of the wall of the cone (for example first wall 23 of first truncated cone 20). The bottom left figure of FIG. 14 shows one example an overlap 26 putting the polygon in a position to expand from a twenty-sided polygon to a twenty-one sided polygon. This bottom left figure is not drawn with exact precision at the overlap and the non-uniformity in the thickness of the overlapping sides of the polygon is not intended to illustrate how thick the sides should be. A reverse contraction of the polygonal shape may also be possible in a similar manner.

As can be appreciated from FIG. 3, in preferred embodiments where the wall or sheet or mantle has overlapping layers/coats, the second or otherwise subsequent truncated cone may function as a de-rotating lever that when placed within the first cone may cause the coats of the mantle to be slidingly rotated in the opposite direction of their initial position. This may allow the initial cone to widen in a controlled manner, either uniformly all along the cone or preferably at the distal tip (or in some other preferred embodiments at the base or other portions) of the dilating cone.

In some preferred embodiments of conic retractor 10, as shown in FIG. 3B, a truncated cone may have overlapping wall edges 28a, 28b, in which the wall 23 or wall edges 28a, 28b of the truncated cone, for example first truncated cone 20, comprises teeth 88 (or ridges) on one edge of the wall 23, for example 28a and may comprise corresponding ridges (or teeth) on the opposing edge of the wall 23, for example 28b in order to lock or hold the teeth into the ridges and thereby maintain the dilated state of the cone in place in locked position. The wall 33 or wall edges of the other truncated cones (30, 40, etc.) used in the cone retractor 10 or methods of the present invention may similarly have teeth and corresponding ridges for mating for locking in the desired dilation configuration.

In some preferred embodiments, the cone that is dilated by another cone, for example first truncated cone 20, may be dilatable without having any slit or wraparound or overlapping surfaces. For example, the material of the cone may be elastic like a diaphragm, or superelastic, or maybe deformable, at least at the distal tip of the cone. This may allow outward pressure from the wider distal tip of the second truncated cone being pushed through the first truncated cone 20 to dilate first truncated cone. The same may be true of the second truncated cone 30 dilated by a third truncated cone or of a third truncated cone dilated by a fourth truncated cone, etc.

As shown in FIG. 14,

The effective portion of all cones in the conic retractor is preferably of the same length, meaning the same axial height, from one truncated cone to the other. This may make it easy to determine the end point of each dilation step. An example of the effective portion is the portion having a longitudinal slit or overlap, the portion that is elastic, or the entire length of the cone, or the distal tip (21, 31, etc.) of the respective cones, which can for example occupy up to half the axial height of the cone.

The second (or otherwise subsequent) truncated cone may have an apex angle that is equal to or sufficiently close to an apex angle of the first (or otherwise previously inserted) truncated cone such that when the second (or otherwise subsequent) truncated cone is dilating the first truncated cone the pressure on the walls of the first (or other previously inserted) truncated cone, from the walls of the second truncated cones, is maintained uniform, at least within one centimeter (and in other preferred embodiments at least within two centimeters (ems) or three ems or four ems or eight ems) of the distal end 22 of the first truncated cone (or in other preferred embodiments, within two centimeters or within three centimeters of distal end 22, or in still other preferred embodiments, within the most distal tenth or fifth or quarter or third or half of the axial height of the first truncated cone 20). This may be because the walls of the respective first and second truncated cones 20, 30 are in contact with each other within for example one centimeter of the distal end 22 of the first truncated cone (or in other preferred embodiments, within 2 ems or within 3 ems of the distal end 22, or in still other preferred embodiments, within the most distal tenth or fifth or quarter or third or half of the axial height of the first truncated cone 20). The maintenance of uniform pressure may allow the previously inserted cone to retain its shape intact and not deform.

For example, the second (or another subsequently inserted) truncated cone 30 may have an apex angle that is equal to the first apex angle within a deviation of 1%, or in other preferred embodiments, may have an apex angle that is equal to the first apex angle within a deviation of 5%, or in other preferred embodiments, may have an apex angle that is equal to the first apex angle within a deviation of 10%, or within a deviation of 25%. The apex angle of the first truncated cone 20 (and of one or more subsequent truncated cones) can differ widely. In one preferred embodiment, the first truncated cone 20 has an apex angle of between 1 degree and 10 degrees. The second truncated cone may also have an apex angle of between 1 degree and 10 degrees. In certain preferred embodiments, the first truncated cone has an apex angle of between 4 and 6 degrees, for example 5 degrees. In other preferred embodiments, the apex angle of the first truncated cone 20 (and of one or more subsequent cones) can be 15, 20, 25, 30, 35, 40 or 45 degrees.

Preferably, the horizontal cross-section of truncated cones that are configured to be situated adjacent to one another in conic retractor 10, for example the first and second truncated cones 20, 30 (and similarly the cross-section of second and third truncated cones 30, 40, etc.), are similar or identical, at least at a distal end of the cones. For example, the first and second truncated cones may each have a cross-section that is symmetrical (for example circular or polygonal). In a preferred embodiment, appreciated from viewing FIG. 2, FIG. 3, the first and second truncated cones have a horizontal cross-section that is circular. In other preferred embodiments, the first and second truncated cones have a horizontal cross-section that is polygonal. In certain preferred embodiments, the polygonal horizontal cross-section of the first and second truncated cones is pyramidal. Nonetheless, in certain preferred embodiments, the horizontal cross-section of the first and second truncated cones 20, 30, or the second and third truncated cones 30, 40 (or the cross-section of third and fourth truncated cones etc. or other pairs of truncated cones that are configured to be situated adjacent to one another), may be dissimilar and each truncated cone in the pair of cones may have different shapes from one another. For example first truncated cone 20 may be circular in cross-section while second truncated cone 30 may be polygonal in cross-section. This also applies to the cross-section and shape of the truncated cones in their initial shape prior to dilation. Furthermore, in certain other preferred embodiments, a truncated cone 20 that has a circular cross-section prior to being dilated by a further truncated cone may have a different cross-section, for example an elliptical cross-section, after dilation by a truncated cone of a different cross-section (for example a cone having an elliptical cross-section), or for other reasons.

Truncated cones of non-symmetrical cross-section or non-symmetrical portions of any truncated cone may also be said to have a “maximum width” at a certain axial distance from the base, for example at the distal end. If the walls of the truncated cone have non-negligible thickness, then the terms “maximum inner width” and “maximum outer width” refer to maximum widths of the truncated cone measured at the inner wall or at the outer wall, respectively. The average maximum inner width and the average maximum outer width refer to the maximum inner width or the maximum outer width within X units of axial distance (toward the base) from the distal end, and this average maximum inner width or average maximum outer width would just be the average of the maximum inner (or outer) widths across the X units of distance from the distal end toward the base as measured along the cone's axial height. Accordingly, first truncated cone 20 may have a first maximum inner width at a distal end of the first truncated cone and second truncated cone 30 may have a second maximum outer width at a distal end of the second truncated cone, the second maximum outer width larger than the first maximum inner width.

As seen from FIGS. 1-12, the first and/or second (and/or third or further) truncated cones 20, 30, 40 may include a handle or other holding/anchoring feature that allows a user to insert the cone into the tissue or into a cone previously inserted into the tissue, or previously inserted into another cone inserted into the tissue. The handle or other anchoring feature may secure the cone against axial displacement while a subsequent cone and/or larger conical dilator is introduced. As can be seen from FIGS. 1-12, the handle may be situated at a proximal end of the cone. In other preferred embodiments, the handle or other anchoring component may be situated on a proximal extension which extends beyond the proximal end of the preceding cones. Accordingly, typically a proximal end of a cone inserted into another cone in accordance with the method or apparatus of the present invention, would be adjacent the proximal end of the cone into which it is inserted. For example, first and second truncated cones 20, 30 may have their proximal ends adjacent one another and their distal ends adjacent one another. Furthermore, a second handle of a second truncated cone may be adjacent or proximal to a proximal end of the first truncated cone or adjacent or proximal to the first handle of the first truncated cone.

Accordingly, as shown in FIG. 13, the present invention may be described as a method 100 of conic retraction of tissue of a mammal. In this case, method 100 may comprise a step 110 of inserting an at least partially hollow first truncated cone into the tissue. The method 100 may also include a step 120 of inserting at least part of a second truncated cone into the first truncated cone such that the second truncated cone widens/dilates at least part of the first truncated cone, for example at least at a distal end of the first truncated cone, or in other preferred embodiments, an entire length, or a longitudinal portion of first truncate cone having a longitudinal slit or in still other preferred embodiments a longitudinal portion of the first truncated cone 20 having overlapping wall edges. The second truncated cone 30 may widen/dilate the at least a distal end of the first truncated cone when an outer wall surface at a distal end of the second truncated cone presses against an inner wall surface at the distal end of the first truncated cone. In some preferred embodiments, the dilation may be accomplished by pushing the distal tip of a second (or otherwise subsequent) cone, which may be wider than the distal tip of the first (or otherwise “previously inserted” cone), into the distal tip of the first or previously inserted cone. The second truncated cone 30 (or other subsequent cone) may or may not be at least partially hollow. The second truncated cone 30 (or other subsequent cone) may have a larger maximum width at its distal end than a maximum width of the first truncated cone at a distal end of the first truncated cone.

Method 100 may have additional steps. For example, method 100 may have a step of inserting the at least part of the second truncated cone into the first truncated cone such that the second truncated cone widens an average maximum inner width of the first truncated cone in a distal tip defined to be within one centimeter (or in other preferred embodiments, two centimeters, or 3, 4, 5, 6, 7, 8, 9, 15, 20, 30, 40, 50, 60, 70, 80, 90 or 100 millimeters) of the distal end of the first truncated cone.

The dilation or widening of first truncated cone 20 (or of second or third or further truncate cones) may occur in set increments. The increments may be dictated by the increase in the diameter of the openings (i.e. the distal tip) of the first (or of subsequent) truncated cone(s). Once the optimal size dilation has been reached, the last cone may serve as an operative conduit for surgical procedures and deployment of implants or devices, instruments or materials (bone, cement, other). After the incremental dilation, the dilated cone may be superfluous and may be pulled out. For example, the first truncated cone 20 may be removed and the second truncated cone 30 may be kept in place in the tissue. In some preferred embodiments, second truncated cone 30 may then be used as a conduit for surgical procedures and deployment of implants, devices or materials whereas in other preferred embodiments, a third truncated cone 40 may then be inserted to dilate or widen second truncated cone 30. In the latter case, the second truncated cone 30 may optionally be removed and discarded. Third truncated cone 40 may be followed up with as many cones as necessary to achieve the optimal dilation of the tissue.

Method 100 may have a step of inserting at least part of a third truncated cone, which may be or may not be at least partially hollow, into an at least partially hollow second truncated cone such that the third truncated cone widens/dilates at least a distal end of the second truncated cone. Method 100 may also have a step of inserting a first dilator (guide wire) into the tissue prior to inserting the first truncated cone 20. Method 100 may also have a step of removing the first dilator after inserting the second truncated cone. After the second truncated cone has dilated the first truncated cone, if optimal dilation has been achieved, method 100 may include a step of inserting a distractor through the second truncated cone into the tissue and distracting the tissue by a first distance. The step may include utilizing a screw as the distractor, the screw for example being inserted into a space between adjacent vertebrae. The vertebrae may then be distracted by the first distance.

As noted, a third truncated cone may be inserted into the second truncated cone. In that case, the third truncated cone may have a similar structural relationship to the second truncated cone that the second truncated cone has to the first truncated cone, i.e. being wider at the tip, having a similar apex angle, etc.

Method 100 may also have a step of inserting at least part of the third truncated cone into the second truncated cone such that the third truncated cone widens/dilates at least a distal end of the second truncated cone and then inserting a second distractor through the third truncated cone into the tissue and distracting the tissue by a second distance.

A step of method 100 may be configuring the second truncated cone to be at least partially hollow and inserting at least part of the at least partially hollow second truncated cone into the first truncated cone such that a longitudinal slit of the second truncated cone is not aligned with a longitudinal slit of the first truncated cone. For example, the longitudinal slit 39 of second truncated cone 30 may be 180 degrees rotationally from the longitudinal slit 29 of the first truncated cone 20. A third truncated cone 30 may have a longitudinal slit 49 that is not aligned with the longitudinal slit 39 of second truncated cone 20. This non-alignment may serve to ensure that there is uniform pressure from the outer wall of a second truncated cone on the inside wall a first truncated cone (or the outer wall of a third truncated cone on the inner wall of a second truncated cone, etc.).

Method 100 may also have a step whereby the second truncated cone widens the at least a distal end of the first truncated cone and dilates the tissue without rubbing longitudinally against the tissue. Rather, the dilation may be from within the previously inserted cone and may be gentle and gradual.

Method 100 may involve a step of inserting the first truncated cone into the tissue at least 2 millimeters deep, or in other preferred embodiments, at least 3 mm, or at least 4 m or 5, 6, 7, 8, 9, 15, 20, 30, 40, 50, 60, 70, 80, 90 or 100 millimeters deep into the tissue.

In some preferred embodiments of method 100 a step or steps of configuring the second cone to be a second truncated cone and to be at least partially hollow and removing the first truncated cone after the second truncated cone widens/dilates the at least a distal end of the first truncated cone and before inserting a distractor through the second truncated cone may be included.

It should be understood that one or more steps of the methods described herein may be combined. Furthermore, any suitable embodiment of conic retractor 10 described herein consistent with the steps of a particular method may be used in any such method.

As shown in FIG. 17, the present invention may also be described as a method 200 of conic retraction of tissue of a mammal wherein the method 200 comprises a step 210 of inserting an at least partially hollow first truncated three-dimensional object into the tissue; and a step 220 of inserting at least part of a second truncated three-dimensional object into the first truncated three-dimensional object such that the second truncated three-dimensional object widens at least part of the first truncated three-dimensional object, wherein each of the first and second truncated three-dimensional objects: has a base at a first end; has an opening at a second end as a result of being truncated, at least partially horizontally, at the second end; is wider at the first end than at the second end; and gradually widens as one moves along a longitudinal axis of the three-dimensional object from the second end to the first end. Although preferably, the gradual widening (or tapering when going from the base to the distal end) may be at a uniform angle, in certain other preferred embodiments, the steepness of the tapering or widening may vary at different longitudinal points along the cone so long as a second truncated cone is able to be at least partially inserted therein and effectuate the dilation of the first truncated cone, as taught in the present invention.

In a different embodiment, second truncated cone 30 may be solid or partially solid and may be configured to widen first truncated cone 20 after being inserted into first truncated cone 20 but second truncated cone would then be removed. The outer wall surface of the second truncated cone may be conical as in the preferred embodiments with a similar or identical apex angle as the first truncated cone. The second truncated cone may be partially or completely solid or may have an inside that although at least partially hollow, is not conical but rather has some other internal shape. If second truncated cone 30 is solid and not hollow, then second wall 33 may be a generally solid block of material having no inner wall surface. In use, the second truncated cone 30 may be removed and optionally a third or further truncated cone may be inserted into first truncated cone. The third or further truncated cone may be hollow or at least partially hollow as in the preferred embodiments, or may be solid or partially solid.

A deviation of 10% from a magnitude of, for example ten, means between nine and eleven.

While the invention has been described with respect to a limited number of embodiments, it will be appreciated that many variations, modifications and other applications of the invention may be made. Therefore, the claimed invention as recited in the claims that follow is not limited to the embodiments described herein.

1. A conic retractor for dilation of tissue of a mammal, the conic retractor comprising:
an at least partially hollow first truncated cone; and
a second truncated cone,
the second truncated cone configured to at least partially enter the first truncated cone and to dilate at least part of the first truncated cone, the second truncated cone at least partially hollow at a distal end of said second truncated cone.

2. The conic retractor of claim 1, wherein the second truncated cone is configured to at least partially enter the first truncated cone and to dilate at least the distal end of the first truncated cone when at least part of an outer wall surface of the second truncated cone at the distal end of the second truncated cone presses against an inner wall surface of the first truncated cone at the distal end of the first truncated conc.

3. The conic retractor of claim 1, wherein the first truncated cone has a first maximum inner width at a distal end of the first truncated cone, and wherein the second truncated cone has a second maximum outer width at the distal end of the second truncated cone, the second maximum outer width larger than the first maximum inner width.

4. The conic retractor of claim 1, wherein the first truncated cone has a first average maximum inner width within one centimeter of a distal end of the first truncated cone, wherein the second truncated cone has a second average maximum outer width within one centimeter of the distal end of the second truncated cone, and wherein the second maximum outer width is larger than the first average maximum inner width.

5. The conic retractor of claim 1, wherein the first truncated cone has a first average maximum inner width within two centimeters of a distal end of the first truncated cone, wherein the second truncated cone has a second average maximum outer width within two centimeters of the distal end of the second truncated cone, and, wherein the second maximum outer width is larger than the first average maximum inner width.

6. The conic retractor of claim 1, wherein the first truncated cone has a first average maximum inner width within three centimeters of a distal end of the first truncated cone, wherein the second truncated cone has a second average maximum outer width within three centimeters of the distal end of the second truncated cone, and wherein the second average maximum outer width is larger than the first average maximum inner width.

7. The conic retractor of claim 1, wherein a first wall of the first truncated cone has a first longitudinal slit in at least a distal end of the first wall.

8. The conic retractor of claim 7, wherein a second wall of the second truncated cone has a second longitudinal slit in at least a distal end of the second wall.

9. The conic retractor of claim 1, wherein a first wall of the first truncated cone has overlapping wall edges in at least at a distal end of the first wall.

10. The conic retractor of claim 9, wherein a second wall of the second truncated cone has overlapping wall edges in at least at a distal end of the second wall.

11. The conic retractor of claim 9, wherein the overlapping wall edges extend through up to half an axial height of the first truncated cone.

12. The conic retractor of claim 9, wherein the overlapping wall edges are not continuous.

13. The conic retractor of claim 1, wherein the second truncated cone has an apex angle that is equal to or sufficiently close to an apex angle of the first truncated cone such that when the second truncated cone is dilating the first truncated cone a pressure on walls of the first truncated cone is uniform, at least within 2 centimeters of the distal end of the first truncated cone.

14. The conic retractor of claim 1, wherein the second truncated cone has an apex angle that is equal to or sufficiently close to an apex angle of the first truncated cone such that when the second truncated cone is dilating the first truncated cone, the walls of the first and second truncated cones are in contact with each other at least within one-centimeters of the distal end of the first truncated cone.

15. The conic retractor of claim 1, wherein the second truncated cone has an apex angle that is equal to the first apex angle within a deviation of 1%.

16. The conic retractor of claim 1, wherein the second truncated cone has an apex angle that is equal to the first apex angle within a deviation of 5%.

17. The conic retractor of claim 1, wherein the second truncated cone has an apex angle that is equal to the first apex angle within a deviation of 10%.

18. The conic retractor of claim 1, wherein the second truncated cone has an apex angle that is equal to the first apex angle within a deviation of 25%.

19. The conic retractor of claim 1, wherein the first truncated cone has an apex angle of between 1 degree and 10 degrees.

20. The conic retractor of claim 19, wherein the second truncated cone also has an apex angle of between 1 degree and 10 degrees.

21. The conic retractor of claim 1, wherein the first truncated cone has an apex angle of between 4 and 6 degrees.

22. The conic retractor of claim 1, wherein the first and second truncated cones each have a cross-section that is symmetrical.

23. The conic retractor of claim 1, wherein the first and second truncated cones have a horizontal cross-section that is circular.

24. The conic retractor of claim 1, wherein the first and second truncated cones have a horizontal cross-section that is polygonal.

25. The conic retractor of claim 24, wherein the polygonal horizontal cross-section of the first and second truncated cones is pyramidal.

26. The conic retractor of claim 1, wherein the second truncated cone includes a handle situated proximal to a proximal end of the first truncated cone.

27. The conic retractor of claim 1, wherein the first and second truncated cones have the same horizontal cross-sectional shape at a distal end.

28. The conic retractor of claim 1, wherein a structure of the second truncated cone is derivable by further truncating a truncated cone of an apex angle and shape of the first truncated cone parallel to a base and thereby yield a truncated cone having an axial height equal to an axial height of the first truncated cone.

29. A method of conic retraction of tissue of a mammal, the method comprising:
inserting an at least partially hollow first truncated cone into the tissue; and
inserting at least part of a second truncated cone into the first truncated cone such that the second truncated cone widens at least part of the first truncated cone, the second truncated cone at least partially hollow at a distal end of said second truncated cone.

30. The method of claim 29, further comprising inserting the at least part of the second truncated cone into the first truncated cone such that the second truncated cone widens/dilates at least at a distal end of the first truncated cone.

31. The method of claim 30, further comprising inserting the at least part of the second truncated cone into the first truncated cone such that the second truncated cone widens an average maximum inner width of the first truncated cone in a distal tip defined to be within one centimeter of the distal end of the first truncated cone.

32. The method of claim 30, further comprising inserting the at least part of the second truncated cone into the first truncated cone such that the second truncated cone widens an average maximum inner width of the first truncated cone in a distal tip distal to be within two centimeters of the distal end of the first truncated cone.

33. The method of claim 30, further comprising the second truncated cone widens/dilates the at least a distal end of the first truncated cone when an outer wall surface of the second truncated cone at the distal end of the second truncated cone presses against an inner wall surface of the first truncated cone at the distal end of the first truncated cone.

34. The method of claim 30, further comprising dilating the first truncated cone by utilizing an at least partially hollow second truncated cone that has a larger maximum width at the distal end of the second truncated cone than an maximum width of the first truncated cone at a distal end of the first truncated cone.

35. The method of claim 30, further comprising inserting at least part of a third truncated cone into the second truncated cone such that the third truncated cone widens/dilates at least the distal end of the second truncated cone, the second truncated cone being at least partially hollow.

36. The method of claim 30, further comprising inserting a first dilator (guide wire) into the tissue prior to inserting the first truncated cone and removing the first dilator after inserting the second truncated cone.

37. The method of claim 30, further comprising inserting a distractor through the second truncated cone into the tissue and distracting the tissue by a first distance.

38. The method of claim 37, further comprising utilizing a screw as the distractor, the screw inserted into a space between adjacent vertebrae, and distracting the vertebrae by the first distance.

39. The method of claim 35, further comprising inserting at least part of the third truncated cone into the second truncated cone such that the third truncated cone widens/dilates at least the distal end of the second truncated cone and then inserting a second distractor through the third truncated cone into the tissue and distracting the tissue by a second distance.

40. The method of claim 30, wherein the second truncated cone is at least partially hollow and further comprising inserting at least part of the at least partially hollow second truncated cone into the first truncated cone such that a longitudinal slit of the second truncated cone is not aligned with a longitudinal slit of the first truncated cone.

41. The method of claim 30, further comprising the second truncated cone widening the at least a distal end of the first truncated cone and dilating the tissue without rubbing longitudinally against the tissue.

42. The method of claim 30, further comprising inserting the first truncated cone into the tissue at least 20 millimeters deep.

43. The method of claim 30, wherein the second truncated cone is at least partially hollow and further comprising removing the first truncated cone after the second truncated cone widens/dilates the at least a distal end of the first truncated cone and before inserting a distractor through the second truncated cone.

44. A method of conic retraction of tissue of a mammal, the method comprising:
inserting an at least partially hollow first truncated three-dimensional object into the tissue; and
inserting at least part of a second truncated three-dimensional object into the first truncated three-dimensional object such that the second truncated three-dimensional object widens at least part of the first truncated three-dimensional object,
each of the first and second truncated three-dimensional objects:
has a base at a first end;
has an opening at a second end as a result of being truncated, at least partially horizontally, at the second end;
is wider at the first end than at the second end;
gradually widens as one moves along a longitudinal axis of the three-dimensional object from the second end to the first end.

45. The conic retractor of claim 1, wherein the second truncated cone has an apex angle that is equal to the first apex angle within a deviation of 25%.
